(19) United States
(12) Patent Application Publication
(10) Pub. No.: US 2020/0360553 A1
(43) Pub. Date: Nov. 19, 2020

(54) AEROSOL COMPOSITION, AND AEROSOL SPRAY CAN FILLED WITH AEROSOL COMPOSITION

(71) Applicant: SHARP KABUSHIKI KAISHA, Sakai City (JP)

(72) Inventors: TAKESHI SATOH, Sakai City (JP); YOSHITAKA KAWASE, Sakai City (JP); TOMOYA TSUTSUMINO, Sakai City (JP)

(21) Appl. No.: 16/837,598

(22) Filed: Apr. 1, 2020

(30) Foreign Application Priority Data
May 13, 2019 (JP) 2019-090652

(51) Int. Cl. A61L 2/22; A01N 31/02; C09K 3/30; A61L 2/26; B65D 83/24

(52) U.S. Cl. CPC A61L 2/22; A01N 31/02; A61L 2202/15; A61L 2/26; B65D 83/24; C09K 3/30

(57) ABSTRACT

The present invention provides an aerosol composition filled in an aerosol spray can and including at least a spray liquid containing water and a functional fine particle, and a propellant containing dimethylether. A volume ratio G/L of a volume L of the spray liquid and a volume G of the propellant in the spray can is 0.9 to 1.5.

### BACKGROUND OF THE INVENTION

### Field of the Invention

The present invention relates to an aerosol composition, and an aerosol spray can filled with the aerosol composition.

### Description of the Background Art

Conventionally, for an aerosol composition to be filled into an aerosol spray can, dimethylether (DME) has been widely used as a propellant. For example, in Japanese Patent No. 4931026, dimethylether is used as a spray type bactericidal decomposition agent for a propellant, this dimethylether is mixed with a photocatalyst (functional fine particle) manufactured based on water containing titanium oxide, and this mixture is filled into a spray can.

Incidentally, in a case of an aerosol spray can, when a jetting button is operated, a functional fine particle such as titanium oxide to be jetted together with an vaporized propellant (dimethylether) is jetted at a predetermined rate per unit time, and as such a state, it is desired to effectively exhibit performances of functions (e.g. sterilization, and the like) of the functional fine particle.

Thus, conventionally, as described in Japanese Patent No. 4931026, it is common that a spray liquid obtained by mixing a functional fine particle with water or the like is mixed with a dimethylether (propellant) to prepare an aerosol composition, and the functional fine particle is relatively uniformly dispersed in the aerosol composition.

However, when a specific gravity of the functional fine particle is high in such an aerosol composition, the functional fine particle in the spray can tends to precipitate in the aerosol composition, and a large amount of the functional fine particle is present at the bottom side of the can. As a result, when jetting the aerosol composition, a jetting rate of the functional fine particle changes with time, and a constant rate-jetting performance for jetting the particle with a predetermined rate is deteriorated. Not only that, when the spray can content is used up, i.e. in a state that the propellant is not jetted even by operating the jetting button, the precipitated functional fine particle remains at the bottom and the bottom periphery of the can, resulting in a disadvantage that the whole amount of the functional fine particle in the can cannot be effectively used up.

Also, when the aerosol spray can is of a so-called whole amount-jetting type (or a one push-use up type) for jetting a whole amount of an aerosol composition in a container during a one-push operation of a jetting button, there is a tendency that a temperature of the spray can decreases as the propellant continues to vaporize during the jetting, therefore a jet pressure of the jet gas gradually decreases as the temperature decreases, a performance as the propellant is deteriorated, and the functional fine particle becomes difficult to jet with time. Thereby, there is a disadvantage that the constant rate-jetting performance of the functional fine particle is deteriorated, and the functional fine particle tends to remain in a large amount in the can after used up with one push.

In view of the aforementioned points, an object of the present invention is to stabilize a functional fine particle in a spray liquid so as to uniformly disperse in dimethylether as a propellant by appropriately setting an intra-can constitution ratio of the propellant containing dimethylether and the spray liquid containing the functional fine particle as an aerosol composition filled in an aerosol spray can, so that a residual amount of the functional fine particle in the can when used up is limited to a small amount while securing a desirable constant rate-jetting performance of the functional fine particle.

### SUMMARY OF THE INVENTION

The aerosol composition according to the present invention is filled in an aerosol spray can, and is characterized in that the aerosol composition includes at least a spray liquid containing water and a functional fine particle, and a propellant containing dimethylether, and a volume ratio GIL of a volume L of the spray liquid and a volume G of the propellant in the aerosol spray can is 0.9 to 1.5.

Herein, the functional fine particle is a metal or a metal oxide having a specific gravity of 4 or higher.

Furthermore, the aerosol spray can according to the present invention is characterized in that the aerosol spray can includes a container filled with the aerosol composition, and a jetting button having a nozzle hole for jetting the aerosol composition, and the jetting button is of a whole amount-jetting type.

With the aerosol composition according to the present invention, the functional fine particle can be stabilized so as to uniformly disperse in the propellant containing dimethylether in the can in a state that the aerosol composition is filled in the aerosol spray can, and furthermore a residual amount of the functional fine particle in the spray can when used up can be limited to a small amount.

In particular, even when the functional fine particle contained in the spray agent is the metal or the metal oxide having the specific gravity of 4 or higher, a desirable uniform dispersibility of the functional fine particle in the can can be secured, and the residual amount of the functional fine particle in the spray can when used up can be limited to a small amount.

In addition, with the aerosol spray can according to the present invention, the residual amount of the functional fine particle in the can after the whole content has been thoroughly jetted can be limited to a small amount even when a jet pressure of a vaporizing filler gradually decreases during operation of the whole amount-jetting type jetting button.

### BRIEF DESCRIPTION OF THE DRAWINGS

FIG. 1 is a longitudinal sectional view of an aerosol spray can filled with an aerosol composition according to an embodiment.

FIG. 2 is a diagram presenting constitutions in Examples 1 to 5 and Comparative Examples 1 and 2, in which a volume ratio of a spray liquid and a propellant in the aerosol composition is varied.

FIG. 3 is a diagram presenting results of a residual amount of a functional fine particle when the spray can content is used up, and results of judging performances, in Examples 1 to 5 and Comparative Examples 1 and 2 in FIG. 2.

### DESCRIPTION OF THE PREFERRED EMBODIMENTS

Hereinafter, embodiments of the present invention will be explained with reference to the figures.

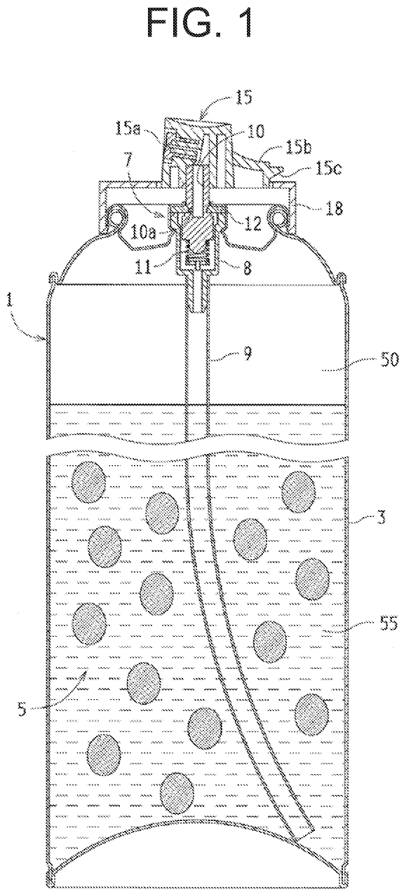

FIG. 1 is a longitudinal sectional view of an aerosol spray can filled with an aerosol composition according to this embodiment.

In FIG. 1, an aerosol spray can 1 has a container 3 having pressure resistance and airtightness, and a container 3 is filled with an aerosol composition 5. A valve 7 for jetting the aerosol composition 5 is attached to an upper end of the container 3, and a jetting button 15 for opening and closing the valve 7 is attached to an upper portion of the valve 7.

In the aforementioned valve 7, a dip tube 9 is connected to a lower end of its valve body housing 8, and a vertically extending hollow stem 10 is energized upward by a spring 11 inside the valve body housing 8. When the jetting button 15 disposed on an upper end of the stem 10 is not pushed down, the stem 10 is energized upward by the spring 11, and a passage hole 10a formed on a lateral side of the stem 10 is closed by a gasket 12 to prevent the aerosol composition 5 from jetting.

On the other hand, when the jetting button 15 is pushed down, the stem 10 moves downward by the pushing operation, the passage hole 10a leaves apart from the gasket 12 and opens, and the aerosol composition 5 in the container 3 moves from a lower end of the dip tube 9 through the valve body housing 8, furthermore moves from the passage hole 10a through an inner space of the stem 10, and is jetted from a nozzle hole 15a formed on a lateral side of the jetting button 15.

The aerosol spray can 1 is of a whole amount-jetting type or a one push-use up type, which makes it possible to jet a whole amount of the aerosol composition 5 in the container 3 when the jetting button 15 is pushed down. Specifically, in the aerosol spray can 1, the jetting button 15 has a projecting portion 15c formed on an extending portion 15b which extends on an outer periphery of the jetting button 15. When the jetting button 15 is pushed down, the projecting portion 15c moves downward, enters an inside of a corner portion of a cover 18 disposed on a shoulder portion of the container 3, and engages with the cover 18. Therefore, after an operator stops pushing down the jetting button 15, the projecting portion 15c maintains the pushed-down state of the jetting button 15, and holds the opening state of the passage hole 10a to continue the jetting of the aerosol composition 5. Incidentally, in the following description, the state that the aerosol composition 5 filled in the container 3 is not jetted from the nozzle hole 15a when the jetting button 15 is pushed down is also referred to as “when the whole content is thoroughly jetted”, “when the whole content is used up”, or “the whole content is used up with one push”.

In a state that the aerosol composition 5 is filled inside the container 3 at a high pressure, a part of a propellant (explained in detail later) 50 evaporates to form a gas phase at normal temperature, as illustrated in FIG. 1. In addition, a mixture 55 of the propellant in a liquid phase and a spray liquid (explained in detail later) is positioned under the gas phase. When the jetting button 15 is pushed down, a gas pressure of the propellant 50 in the gas phase raises a part of the mixture 55 through the dip tube 9 into the valve body housing 8, and then the mixture 55 is discharged from the nozzle hole 15a of the jetting button 15 through the passage hole 10a and the inner space of the stem 10 to the outside. At this time, the propellant in the discharged mixture 55 rapidly expands around the nozzle hole 15a, and therefore the mixture 55 in a mist form is spouted to the outside.

### Constitution of Aerosol Composition

Hereinafter, a constitution of the aerosol composition 5 in the container 3 will be explained.

The aerosol composition 5 is composed of a spray liquid and an aerosol propellant. The spray liquid is composed of the functional fine particle, water, and ethanol. The aerosol propellant is composed only of dimethylether (DME).

Examples of the functional fine particle in the spray liquid include a photocatalyst fine particle, and a metal fine particle used for a deodorant, a bactericide, a fungicide, or the like. The functional fine particle includes a metal or a metal oxide. Examples of the metal include gold, silver, copper, platinum, and zinc, and examples of the metal oxide include tungsten oxide, and titanium oxide. The functional fine particle may be particularly a metal or a metal oxide having a specific gravity of 4 or higher. The photocatalyst fine particle is e.g. tungsten oxide. Tungsten oxide is excellent in responsiveness to photocatalytic reaction in a visible light wavelength band. Incidentally, an auxiliary catalyst for the catalytic action may be added to the photocatalyst, and also an organic component having functions of the deodorant, the bactericide, and the fungicide may be added to the metal fine particle used for the deodorant, the bactericide, the fungicide, or the like.

Ethanol contained in the spray liquid has good wettability and good compatibility with the aerosol propellant (dimethylether). Thus, the functional fine particle contained in the spray liquid can be uniformly dispersed in the whole aerosol composition 5, and uniform dispersibility can be desirably secured. Note that FIG. 1 exaggeratedly illustrates the functional fine particles in a large elliptical shape, and illustrates a state that those functional fine particles uniformly disperse in the whole aerosol composition 5 (in the liquid phase).

When manufacturing a spray liquid, first, the functional fine particle is dispersed in water using a general wet type disperser, e.g. an ultrasonic disperser, a colloid mill, a bead mill, or the like to prepare a slurry liquid. Then the slurry liquid is mixed with ethanol using a general liquid mixer equipped with stirring blades to manufacture a spray liquid.

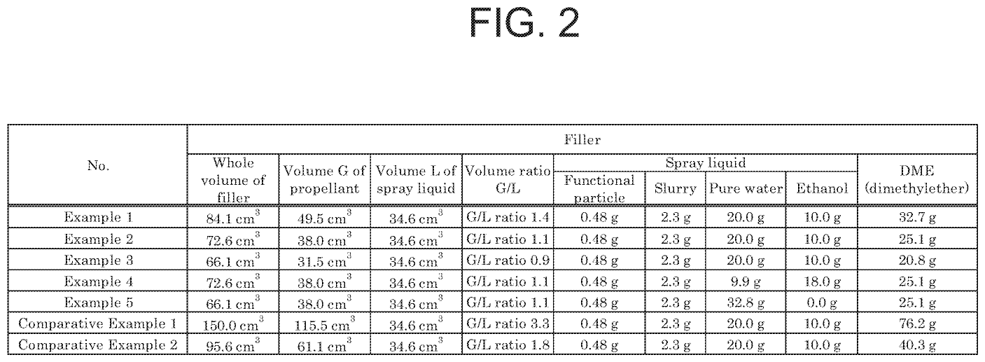

Constitution Ratio of Spray Liquid and Aerosol Propellant A constitution ratio of the spray liquid and the aerosol propellant (dimethylether) in the aerosol composition 5 filled in the container 3 is set so that, when a volume of the spray liquid is represented by L and a volume of the dimethylether is represented by G, a volume ratio G/L is 0.9 to 1.5. This volume ratio G/L is determined based on measurement results presented in FIG. 2 so that a residual amount of the functional fine particle in the container 3 is not more than a predetermined amount when the whole content of the aerosol composition 5 in the container 3 is used up.

FIG. 2 presents a diagram in which the constitution ratio of the spray liquid and the aerosol filler in the aerosol composition 5 filled in the aerosol spray can 1 is varied among five types.

FIG. 2 presents five examples and two comparative examples. In Example 1, 0.48 g of the functional fine particle is dispersed in a ratio of 21 wt % in pure water to prepare 2.3 g of slurry liquid, which is mixed with 20.0 g of pure water and 10.0 g of ethanol to prepare a spray liquid, this spray liquid is filled into the aluminium spray can 1 having a liquid volume capacity of 220 ml. In this state, the valve 7 is clinched, and then, as an aerosol propellant, 32.7 g of dimethylether alone is filled in the spray can. In Examples 2, 4 and 5, the amount of the filled propellant (dimethylether) in Example 1 is changed to 25.1 g. Additionally, in Example 3, the amount of the filled propellant in Example 1 is changed to 20.8 g. Furthermore, in Example 4, a spray liquid is produced by blending 9.9 g of pure water and 18.0 g of ethanol into 2.3 g of the slurry liquid in Example 1, and in Example 5, a spray liquid without ethanol is produced by blending 32.8 g of pure water alone into 2.3 g of the slurry liquid. Thus, in Examples 1 to 5, the volumes L of the spray liquids are the same (34.6 cm³), but the volumes G of the propellants are different from each other, the volumes increase in descending order of Example 1, Examples 2, 4, 5, and Example 3 (49.5 cm³, 38.0 cm³, 38.0 cm³, 38.0 cm³, 31.5 cm³). That means, the volume ratio G/L of the spray liquid and the propellant is set to 1.4 in Example 1, to 1.1 in Examples 2, 4, 5, and to 0.9 in Example 3.

On the other hand, in Comparative Example 1, the amount of the filled propellant in Example 1 is changed to 76.2 g, and in Comparative Example 2, the amount of the filled propellant in Example 1 is changed to 40.3 g. Thus, the volume G of the propellant in Comparative Example 1 is larger than in Comparative Example 2, and the volume ratio G/L of the spray liquid and the propellant is set to 3.3 in Comparative Example 1, and to 1.8 in Comparative Example 2.

Incidentally, in FIG. 2, the volumes of the whole fillers in the aerosol compositions 5 are also described as reference. In addition, FIG. 2 presents cases that a content ratio of ethanol in the spray liquid is fixedly set to about 30 wt % (=10/(2.3+20+10) in all of Examples 1 to 3 and Comparative Examples 1 and 2, and to about 60 wt % (=18/(2.3+9.9+18) in Example 4, and to 0 wt % in Example 5, as examples. Furthermore, in Examples 1 to 5 and Comparative Examples 1 and 2, a photocatalytic fine particle containing tungsten oxide having a heavy specific gravity is used as the functional fine particle.

In Examples 1 to 5 and Comparative Examples 1 and 2, when filling the aerosol composition 5 into the spray can 1, first, the spray liquid is filled into the container 3 at atmospheric pressure, and in this state, the valve 7 is inserted into the container 3, and clinched to fix the valve 7 to the upper end of the container 3. Then, only a predetermined amount of liquid dimethylether as the propellant is filled into the container 3 via the stem 10 at a high pressure. Finally, the jetting button 15 is attached to the container 3 to complete the aerosol spray can 1.

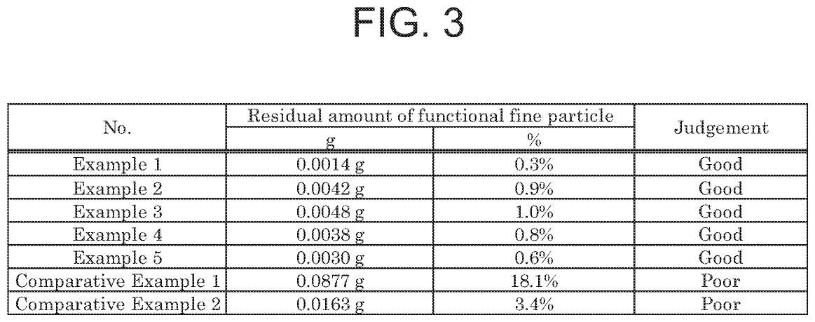

FIG. 3 presents residual amounts of the functional fine particle when the whole content in the spray can 1 is used up, in Examples 1 to 5 and Comparative Examples 1 and 2.

The method for measuring the residual amount of the functional fine particle is as follows. First, the spray can 1 after used up with one push is opened, and the liquid remaining in the can is collected in a dish. In addition, deposits on the wall surface inside the can are also collected in the dish using a small amount of water. Then, the dish is heated on a hot plate to vaporize water. Subsequently, the dish is sufficiently cooled, then a weight of the whole dish is measured, and a difference between the whole weight and the weight of only the dish is defined as the residual amount (solid weight) of the functional fine particle in the container 3.

FIG. 3 presents the residue weights (unit: g) of the functional fine particle in the container 3, and percentages (%) of the residue with respect to the whole weight (0.48 g) of the functional fine particle blended in the spray liquid in Examples 1 to 5 and Comparative Examples 1 and 2. In addition, in FIG. 3, as results of judging the residue amounts of the functional fine particle, a case with a small and desirable residual amount of the functional fine particle is judged as Pass and marked as “Good”, and a case with a large residual amount of the functional fine particle is judged as Failure (Reject) and marked as “Poor”. In this judgement, a case that the residual amount of the functional fine particle with respect to the whole weight of the functional fine particle is lower than e.g. a criterion 2% is judged as Pass. The criterion 2% is assumedly at such a level that the remaining functional fine particle is invisible at the bottom periphery and the like of the container 3 as places where a lot of functional fine particle remains.

As can be seen from FIG. 3, in Examples 1, 2, 3, 4 and 5 with the residual amount of 1% or lower, the residual amounts of the functional fine particle surpass the Pass level, and in Comparative Examples 1 and 2 with the residual amount exceeding 3% is judged as Reject.

Among Examples 1 to 5 at levels surpassing the Pass level, Example 3 shows the largest residual amount, and the volume ratio G/L of 0.9. The state of the volume ratio G/L=0.9 or lower is unsuitable as the aerosol spray can, because the volume of dimethylether is small, and a spraying force of dimethylether is not enhanced during the jetting from the nozzle hole 15a. Thus, the value of the volume ratio G/L for reducing the residual amount of the functional fine particle is at least 0.9.

On the other hand, among Examples 1 to 5 at levels exceeding the Pass level, Example 1 shows the smallest residual amount (residual amount=0.3%), and Example 1 shows the volume ratio G/L of 1.4. Thus, in Comparative Example 2 (volume ratio G/L=1.8) and Comparative Example 1 (volume ratio G/L=3.3) having the volume ratio G/L slightly larger than of Example 1, the residual amount of the functional fine particle drastically increases (3.4% in Comparative Example 2, 18.1% in Comparative Example 1). Consequently, the volume ratio G/L of about 1.5 slightly higher than 1.4 in Example 1 is an upper limit which makes it possible to reduce the residual amount of the functional fine particle. It is assumed that when the volume ratio G/L exceeds this upper limit (G/L=1.5), the residual amount of the functional fine particle drastically increases.

Thus, as described above, in the aerosol composition containing at least water and the functional fine particle as the spray liquid and containing dimethylether as the propellant, when the volume ratio G/L of the spray liquid and the propellant filled in the spray can 1 is set to 0.9 to 1.5, the residual amount of the functional fine particle in the can when used up can be limited to a very small amount.

As described above, it is considered that the reason why the residual amount of the functional fine particle can be limited to a very small amount by setting the volume ratio G/L to 0.9 to 1.5 is because the spray liquid (functional fine particle and pure water) and the propellant (dimethylether) are sufficiently mixed in the container 3 of the spray can 1 under the condition of the aforementioned volume ratio G/L, and the functional fine particle is uniformly dispersed in the mixture. Thus, when the jetting button 15 is pushed down, the functional fine particle is constantly jetted at a predetermined rate per unit time regardless of the lapsed time after the push-down, so that the constant rate-jetting performance of the functional fine particle can be desirably secured to desirably exhibit the expected performance of the functional fine particle during the jetting.

In addition, even when tungsten oxide having a high specific gravity is used as the functional fine particle included in the slurry liquid, the tungsten oxide can be uniformly dispersed in the aerosol composition 5, and therefore also when a metal or a metal oxide having a high specific gravity e.g. 4 or higher as the functional fine particle is blended into the slurry liquid, the residual amount of the functional fine particle in the spray can 1 when used up can be limited to a very small amount while securing the desirable constant rate-jetting performance of the functional fine particle of the metal or the metal oxide.

As for the functional fine particle contained in the spray liquid, a volume average particle diameter (50% particle diameter) D50 of the particle dispersed in the spray liquid is desirably 500 nm or smaller. In a case where the D50 is 500 nm or smaller, the functional fine particle can be uniformly dispersed in the spray liquid, and where the D50 is larger than 500 nm, the uniform dispersibility decreases. Thus, since the uniform dispersibility of the functional fine particle in the spray liquid can be further improved by setting the 50% particle diameter D50 of the functional fine particle to 500 nm or smaller, the constant rate-jetting performance of the functional fine particle when pushing down the jetting button 15 can be further improved. Particularly, in case where the 50% particle diameter D50 of the functional fine particle is further set to 250 nm or smaller, the uniform dispersibility of the functional fine particle in the spray liquid can be maintained over a long time, and therefore the spraying performance is expected to be further improved.

When the spray liquid contains ethanol as in Examples 1 to 4, this ethanol has a high compatibility with dimethylether (propellant). Thus, when the spray liquid obtained by mixing the functional fine particle with water and ethanol is mixed with dimethylether (propellant) to constitute the aerosol composition, the functional fine particle can be more uniformly dispersed in the aerosol composition.

Furthermore, when ethanol is contained in the spray liquid, a content rate of ethanol is desirably set to 60 wt % or lower. In a case of 60 wt % or lower, the uniform dispersibility of the functional fine particle in the spray liquid is desirable, but in a large amount of ethanol at a content rate exceeding 60 wt %, the uniform dispersibility of the functional fine particle decreases and the functional fine particle readily precipitates in the container 3. Thus, the content rate of ethanol in the spray liquid set to 60 wt % or lower makes it possible to further improve the uniform dispersibility of the functional fine particle in the spray liquid to further improve the constant rate-jetting performance of the functional fine particle when pushing down the jetting button 15.

Furthermore, in the case of the whole amount-jetting type (one push-use up type) aerosol spray can 1, when pushing down the jetting button 15, the projecting portion 15c engages with the corner portion of the cover 18, and the aerosol composition 5 in a mist form continues to be jetted from the nozzle hole 15a. In this state, since a temperature of the spray can 1 gradually decreases as the propellant (dimethylether) continues to vaporize, there is a tendency that a jet gas pressure of the vaporizing propellant also decreases with time, the functional fine particle in the aerosol composition 5 is not jetted well together with the jet gas, and the residual amount of the functional fine particle in the can also increases. However, as described above, this embodiment takes the configuration that the volume ratio G/L of the spray liquid and the propellant (dimethylether) is set to 0.9 to 1.5 to secure a desirable constant rate-jetting performance of the functional fine particle, and therefore, even with a whole amount-jetting type aerosol spray can 1, the residual amount of the functional fine particle in the can when used up with one push can be limited to a very small amount compared to background art.

In this embodiment, in Examples 1 to 5 and Comparative Examples 1 and 2, when 0.48 g of the functional fine particle was contained in the aerosol composition 5, the weight (g) of the remaining functional fine particle in the can was determined as presented in FIG. 3, but, needless to say, the weight of the contained functional fine particle may be set to a large amount or a small amount. In this case, the weight (g) of the remaining functional fine particle in the can is large or small, but it is considered that since the weight of the remaining functional fine particle in the can is measured in percentage terms as presented in FIG. 3, the residual amount (%) of the functional fine particle in the can is determined as almost the same percentage value regardless of the weight of the contained functional fine particle.

Additionally, in this embodiment, the case that the aerosol composition 5 is filled in the whole amount-jetting type aerosol spray can 1 has been described as an example, but, needless to say, the present invention may be applied to a normal type aerosol spray can in which once stopping the push-down operation of the jetting button 15, the jetting is stopped.

The present invention can be executed in various other forms without departing from the spirit or main characteristics of the present invention. Thus, the aforementioned embodiments are merely examples, and should not be limitedly interpreted. All modifications and changes belonging to the scope of equivalents for claims in the present invention come within the scope of the present invention.

### INDUSTRIAL APPLICABILITY

The present invention is useful as an aerosol composition to be filled in an aerosol spray can, because a residual amount of a functional fine particle in the spray can when used up can be limited to a small amount while sufficiently securing an expected performance of the functional fine particle contained in a spray liquid during the jetting.

### DESCRIPTION OF REFERENCE NUMERALS

1: Aerosol spray can
3: Container
5: Aerosol composition
15: Jetting button
15a: Nozzle hole
15c: Projecting portion

What is claimed is:

1. An aerosol composition charged in an aerosol spray can, the aerosol composition including at least a spray liquid containing water and a functional fine particle, and a propellant containing dimethylether, wherein
a volume ratio G/L of a volume L of the spray liquid and a volume G of the propellant in the aerosol spray can is 0.9 to 1.5.

2. The aerosol composition according to claim 1, wherein
the functional fine particle is a metal or a metal oxide having a specific gravity of 4 or higher.

3. The aerosol composition according to claim 2, wherein
the metal oxide is a photocatalytic fine particle containing tungsten oxide.

4. The aerosol composition according to claim 1, wherein
the volume average particle diameter D50 of the functional fine particle is 500 nm or smaller.

5. The aerosol composition according to claim 1, wherein
the spray liquid further contains ethanol, and
a content rate of ethanol in the spray liquid is 60 wt % or lower.

6. An aerosol spray can comprising:
a container filled with the aerosol composition according to claim 1, and
a jetting button having a nozzle hole that jets the aerosol composition.

7. The aerosol spray can according to claim 6, wherein
the jetting button is of a whole amount-jetting type.
